(19) United States
(12) Patent Application Publication
(10) Pub. No.: US 2007/0191767 A1
(43) Pub. Date: Aug. 16, 2007

(54) Bifurcated Catheter Joints

(75) Inventors: Joe Hennessy, Ballybrit (IE); Ashish Varma, Ballybrit (IE); Robert Murray, Santa Rosa, CA (US); Gerry Clarke, Ballybrit (IE); Terry Guinan, Ballybrit (IE); Patrick Duane, Ballybrit (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/695,697

(22) Filed: Apr. 3, 2007

Related U.S. Application Data
(63) Continuation-in-part of application No. 11/303,755, filed on Dec. 16, 2005.

(51) Int. Cl. A61M 29/00; A61M 31/00; A61M 37/00

(52) U.S. Cl. 604/103.04

(57) ABSTRACT

A rapid exchange catheter includes an exchange joint that is coupled between a proximal shaft and first and second distal shafts. The exchange joint includes a proximal end that is configured to be coupled to the proximal shaft, and a distal end that is configured to be coupled to the first distal shaft and the second distal shaft. The distal end includes a first portion that includes a first guidewire lumen, and a second portion that includes a second guidewire lumen. The exchange joint also includes a guidewire port that is configured to provide access for a first guidewire into the first distal inner lumen via the first guidewire lumen and a second guidewire into the second distal inner lumen via the second guidewire lumen.

### CROSS-REFERENCE TO RELATED APPLICATIONS

This application is a continuation-in-part of U.S. patent application Ser. No. 11/303,755, filed on Dec. 15, 2005, entitled “RAPID EXCHANGE CATHETER HAVING A UNIFORM DIAMETER EXCHANGE JOINT,” and currently pending, the content of which is hereby incorporated by reference in its entirety.

### TECHNICAL FIELD

The present invention generally relates to catheters used in the vascular system, and more particularly relates to systems for facilitating exchange of such catheters and associated guidewires, and for using such catheters and guidewires to access selected sites within a patient.

### BACKGROUND

Catheters are inserted into various locations within a patient for a wide variety of purposes and medical procedures. Catheter insertion typically requires the use of a guidewire, particularly when the catheter carries a stent or other relatively bulky therapeutic device. The guidewire may be inserted into a patient's vasculature through the skin, and advanced to the treatment location. Alternatively, the guidewire and the delivery catheter may be advanced together, with the guidewire protruding from the catheter distal end. In either case, the guidewire guides the delivery catheter to the treatment location.

There are various types of catheters, one of which is the “rapid exchange” (RX) or single operator catheter, which is formed with a relatively short guidewire lumen that extends through a short distal catheter segment. The guidewire proximal exit port is typically located about 5 cm to about 30 cm from the catheter distal end. During use, the guidewire is initially placed in the patient's vascular system, and the catheter distal segment is then threaded onto the guidewire. The catheter can be advanced alongside the guidewire with its distal segment being attached to and guided along the guidewire. The catheter can be removed and exchanged for another RX catheter without the need for a relatively long exchange guidewire and without withdrawing the initially placed guidewire.

A cross sectional longitudinal view of one type of RX catheter 50 is depicted in FIG. 1. The RX catheter 50 includes an elongate distal shaft 56 joined to transition tubing 52. The distal shaft 56 includes a coaxial inner guidewire lumen 54 extending to the shaft distal end 53. The transition tubing 52 joins the distal shaft 56 to a proximal shaft 51, which may include or function as an inflation lumen through which a fluid is transported to inflate a balloon 55 when a therapeutic procedure is performed using the RX catheter 50. FIG. 2 is a cross-sectional longitudinal view of an exchange joint 60 where the distal shaft 56 and the transition tubing 52 are joined. As depicted, the transition tubing 52 is inserted into the distal shaft 56. The guidewire lumen 54 is situated alongside the transition tubing at the position where the transition tubing 52 is inserted. During use, the transition tubing 52 transports fluid from the proximal shaft 51 to a distal shaft inflation lumen 57 that is coaxial with the guidewire lumen 54. Thus, the exchange joint 60 effectively transitions the inflation and guidewire lumens into the distal shaft 56 from a proximal side-by-side arrangement to a distal coaxial arrangement.

Assembly of the exchange joint 60 is a somewhat intricate and inefficient process because of the number of components that are bonded together. The assembly process includes flaring the inner diameter of the distal shaft 56 to allow room for insertion of the transition tubing 52, which also may require skiving to minimize the space taken by the transition tubing 52 inside the distal shaft 56. At some point prior to bonding, mandrels are inserted into the guidewire lumen 54 and into the transition tubing 52 in order to prevent their respective passageways from collapsing. FIGS. 3 and 4 are cross sectional views of the exchange joint 60 taken along line A-A in FIG. 2 before and after performing a bonding procedure, with a D mandrel 59 loaded into the transition tubing and a round mandrel 58 loaded into the guidewire lumen 54. The bonding process includes wrapping heat shrink material around the exchange joint 60. Heat is then applied to the exchange joint 60 as the heat shrink material compresses the joint components and brings the joint 60 to the bonded form depicted in FIG. 4.

In addition to its inherently intricate assembly process, the formed exchange joint 60 gives the overall RX catheter a distinctively stepped shape as seen when viewing the joint 60 in FIG. 2. The step in the exchange joint 60 adds bulk to the shaft diameter, which may detrimentally affect catheter performance. More particularly, if the RX catheter 50 exits the distal end of a guide catheter, the step in the exchange joint 60 may become caught on the guide catheter edge while withdrawing the RX catheter from the vessel.

Recent improvements to RX catheters have simplified their exchange joints. For example, FIG. 5 is a cross-sectional perspective view of an exchange joint 70 disclosed in International Publication No. WO 2005/021080. The joint 70 is a unitary molded structure that includes a guidewire port 62 through which a guidewire is introduced into a guidewire lumen 64 inside a distal shaft 66. The joint 70 is tailored at its proximal end 65 for bonding to a proximal shaft 68, and is further tailored at its distal end for bonding to the distal shaft 66. The guidewire port 62 is also tailored for bonding to the guidewire lumen 64 in a manner that produces a side-by-side arrangement between the guidewire lumen 64 and an inflation lumen 69 in the distal shaft 66. Although the molded joint 70 greatly simplifies the overall exchange joint construction, the side-by-side arrangement of the guidewire lumen 64 and the inflation lumen 69 produces a relatively bulky distal shaft 66. Further, the molded joint proximal end 65 is formed around the outer surface of the proximal shaft 68, producing a step that may become caught on a guide catheter edge while withdrawing the catheter from the vessel.

Moreover, the construction of an exchange joint for a bifurcated catheter is even more complicated and time consuming, as such an exchange joint connects a single proximal shaft to two distal shafts, each of which include a guidewire lumen. Such bifurcated catheters may be used for drug eluding bifurcated stent delivery, which utilize two guidewires.

Over the wire (“OTW”) bifurcated stent delivery systems or balloons are systems that are designed to treat bifurcations. Such systems include a distal section that has two balloons and allows for stent delivery or inflation of both a main branch and a side branch at the same time. Simultaneous delivery to both branches makes the procedure more effective and efficient. In an OTW system, the joint between the proximal shaft and the distal bifurcated section can be extremely complicated, particularly when multi-lumen shafts are used, and the proximal shaft and distal section are not made from the same material.

Accordingly, it is desirable to provide an RX catheter that includes an exchange joint that has a comparatively low profile and a substantially uniform outer diameter throughout the joint and at interfaces between the joint and the lumens that the joint brings together. In addition, it is desirable to provide an RX catheter and bifurcated catheter that is simple and efficient to assemble. It is also desirable to provide a joint for connecting a proximal shaft to a distal bifurcated section in an OTW bifurcate stent delivery system that simplifies the connection.

Furthermore, other desirable features and characteristics of the present invention will become apparent from the subsequent detailed description and the appended claims, taken in conjunction with the accompanying drawings and the foregoing technical field and background.

### BRIEF SUMMARY

It is an aspect of the present invention to provide an RX bifurcated catheter that is simple and efficient to assemble.

According to one embodiment of the invention, a rapid exchange catheter is provided. The rapid exchange catheter includes a proximal shaft, which includes a proximal inflation lumen, a first distal shaft, which includes a first distal inner lumen and a first distal outer inflation lumen, a second distal shaft, which includes a second distal inner lumen and a second distal outer inflation lumen, and an exchange joint that is coupled between the proximal shaft and the first and second distal shafts. The exchange joint includes a proximal end that is configured to be coupled to the proximal shaft, and a distal end that is configured to be coupled to the first distal shaft and the second distal shaft. The distal end includes a first portion that includes a first guidewire lumen, and a second portion that includes a second guidewire lumen. The exchange joint also includes a guidewire port that is configured to provide access for a first guidewire into the first distal inner lumen via the first guidewire lumen and a second guidewire into the second distal inner lumen via the second guidewire lumen.

According to an embodiment, a method for manufacturing a bifurcated stent delivery system is provided. The method includes inserting an exchange joint into a mold. The exchange joint includes a proximal end, a distal end, and a guidewire port. The distal end includes a first portion that includes a first lumen, and a second portion that includes a second lumen. The guidewire port is configured to provide access for a first guidewire into the first lumen and a second guidewire into the second lumen. The method also includes insert molding a proximal shaft to the proximal end of the exchange joint. The proximal shaft includes a lumen configured to communicate fluid. The method further includes insert molding a first guidewire lumen to the first portion of the distal end of the exchange joint so that the first guidewire lumen is connected to the first lumen of the exchange joint, and insert molding a second guidewire lumen to the second portion of the distal end of the exchange joint so that the second guidewire lumen is connected to the second lumen of the exchange joint.

It is another aspect of the present invention to provide an OTW bifurcate stent delivery system that is simple and efficient to assemble.

According to another embodiment of the invention, an over-the-wire bifurcate stent delivery system is provided. The over-the-wire bifurcate stent delivery system includes a proximal shaft that includes a first lumen configured to receive a wire, a second lumen configured to receive a wire, and a third lumen configured to receive a fluid. The system also includes a distal section that includes a first distal inner lumen, a second distal inner lumen, and an outer lumen. The system further includes a joint coupled between the proximal shaft and the distal section. The joint includes a proximal portion and a distal portion. The distal portion is configured to receive a proximal end of the distal section, and the proximal portion is configured to receive a distal end of the proximal shaft so that the first lumen in the proximal shaft is connected to the first distal inner lumen, the second lumen in the proximal shaft is connected to the second distal inner lumen, and the third lumen of the proximal shaft is connected to the outer lumen.

According to an embodiment, a joint for coupling a proximal shaft to a distal section of an over the wire bifurcate stent delivery system is provided. The proximal shaft includes a plurality of lumens, and the distal section includes a plurality of lumens. The joint includes a distal portion that is configured to receive a proximal end of the distal section, and a proximal portion that is configured to receive a distal end of the proximal shaft so that a first lumen in the proximal shaft is connected to a first distal inner lumen in the distal section, a second lumen in the proximal shaft is connected to a second distal inner lumen in the distal section, and a third lumen of the proximal shaft is connected to an outer lumen in the distal section.

### BRIEF DESCRIPTION OF THE DRAWINGS

The present invention will hereinafter be described in conjunction with the following drawing figures, wherein like numerals denote like elements, and

FIG. 1 is a cross-sectional longitudinal view of an RX catheter;

FIG. 2 is a cross-sectional longitudinal view of an exchange joint from the RX catheter depicted in FIG. 1;

FIG. 3 is a cross-sectional view of the exchange joint depicted in FIG. 1 before performing a bonding step, the view taken along line 5-5;

FIG. 4 is a cross- sectional view of the exchange joint depicted in FIG. 3 after performing a bonding step;

FIG. 5 is a cross-sectional perspective view of a molded exchange joint in an RX catheter;

FIG. 6 is a perspective view of a unitary exchange joint for an RX catheter according to an embodiment of the invention;

FIG. 7 is an end view of the distal end of the unitary exchange joint depicted in FIG. 6;

FIG. 8 is an end view of the proximal end of the unitary exchange joint depicted in FIG. 7;

FIG. 9 is a perspective view of a unitary exchange joint for an RX catheter according to another embodiment of the invention;

FIG. 10 is a cross-sectional longitudinal view of the unitary exchange joint depicted in FIG. 9, in conjunction with an RX catheter proximal shaft and distal shaft, and further in conjunction with a guidewire lumen;

FIG. 11 is a perspective view of the unitary exchange joint depicted in FIG. 9, the view taken from the joint distal end;

FIG. 12 is a perspective view of the unitary exchange joint depicted in FIG. 9, the view taken from the joint proximal end;

FIG. 13 is a perspective view of a stepped crescent-shaped mandrel according to an embodiment of the invention;

FIG. 14 is a cross-sectional longitudinal view of a multi-component RX catheter exchange joint, including a proximal shaft, a distal shaft, a hypotube functioning as an inflation lumen, a guidewire lumen, and mandrels that are inserted into the inflation lumen and the guidewire lumen during a bonding process;

FIG. 15 is a cross-sectional longitudinal view of a unitary RX catheter exchange joint, a nanotube that functions as a guidewire lumen, a distal shaft, and a proximal shaft depicted to illustrate their relative configuration for a bonding assembly using a pair of illustrated mandrels;

FIG. 16 is a cross-sectional view of the exchange joint depicted in FIG. 14 after performing a bonding procedure, the exchange joint including mandrels in the guidewire lumen and the inflation lumen, the view taken along line 16-16

FIG. 17 is a top view of a multi-component RX bifurcated catheter exchange joint according to an embodiment of the invention;

FIG. 18A is a cross-sectional view of the exchange joint depicted in FIG. 17, the view taken along line 18A-18A;

FIG. 18B is a cross-section view of another embodiment of the exchange joint of FIG. 18A;

FIG. 19 is an end view of the exchange joint shown in FIG. 17, the view taken from the joint distal end;

FIG. 20 is an end view of the exchange joint shown in FIG. 17, the view taken from the joint proximal end;

FIG. 21 is a cross-sectional view of the exchange joint of FIG. 17, the view taken along line 21-21;

FIG. 22 is a cross-sectional view of the exchange joint of FIG. 17, the view taken along line 22-22;

FIG. 23 is a cross-sectional view of an embodiment of a mold for insert molding the exchange joint;

FIG. 24 is a top view of a portion of an OTW bifurcate stent delivery system having a distal section, a proximal shaft, and a joint connecting the distal section to the proximal shaft according to an embodiment of the invention;

FIG. 25 is a perspective exploded view of the joint of the OTW bifurcate stent delivery system of FIG. 24;

FIG. 26 is a distal end view of the joint shown in FIG. 25;

FIG. 27 is a proximal end view of the joint shown in FIG. 25;

FIG. 28 is a cross-section view of the joint of FIG. 26 taken along line 28-28; and

FIG. 29 is a cross-section view of the stent delivery system of FIG. 24 taken along line 29-29.

### DETAILED DESCRIPTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

The present invention includes an RX catheter having an exchange joint in the catheter distal region. The exchange joint may either be a unitary structure or a combination of components as in the previously-described examples, and may have a substantially uniform outer diameter due to a compact arrangement of a plurality of lumens. The RX catheter is also efficiently assembled during a catheter assembly procedure using the exchange joint.

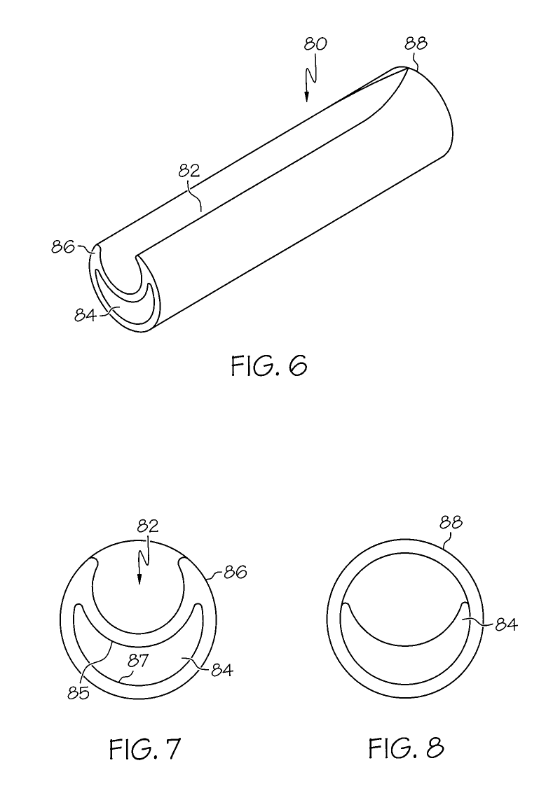

FIG. 6 is a perspective view of an exemplary unitary exchange joint 80 having a substantially uniform outer diameter. FIGS. 7 and 8 are end views of the exchange joint 80 from the joint distal and proximal ends 86 and 88, respectively. The exchange joint 80 includes a guidewire port 82 that provides external access to a guidewire lumen in an RX catheter distal shaft. A guidewire is directed into the guidewire lumen by inserting the guidewire into the guidewire port 82 and feeding it through the exchange joint 80. The guidewire port 82 begins proximate to the exchange joint proximal end 88 and gradually forms a deepening trench in the exchange joint outer surface. At the exchange joint distal end 86, the guidewire port 82 almost entirely encloses a guidewire, and the wall defining the guidewire port consequently has a C-shaped, nearly circular cross-section. An inflation lumen 84 is also included in the exchange joint, and transitions from a substantially circular proximal cross sectional shape to a substantially crescent-shaped distal cross sectional shape. Since the exchange joint is a molded structure, it can be mass manufactured. Further, the unitary joint structure enables quick assembly of an RX catheter.

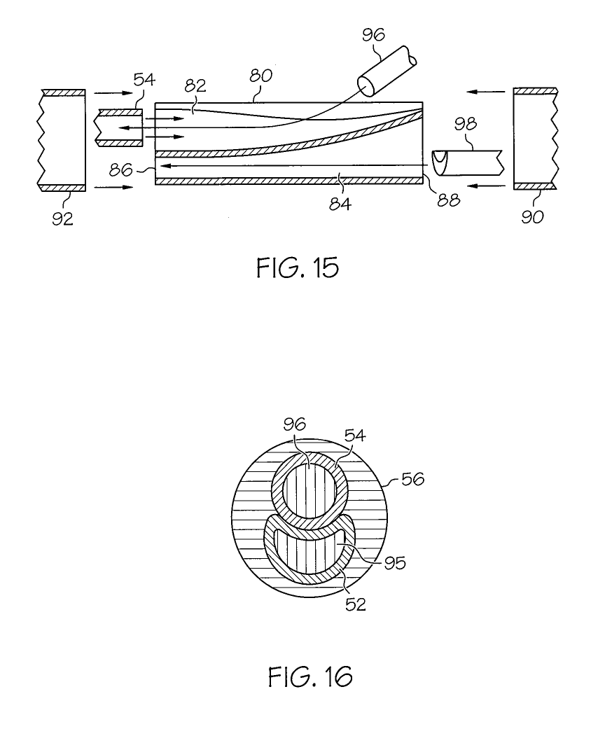

Turning to FIG. 15, a cross-sectional longitudinal view of the exchange joint 80, a guidewire lumen 54, a distal shaft 92, and a proximal shaft 90 are depicted to illustrate their relative configuration for a bonding assembly using a pair of mandrels 96, 98. The proximal end of the guidewire lumen 54 is inserted into the guidewire port 82, and a wire mandrel 96 is inserted into the guidewire lumen 54 to prevent the hypotube from collapsing when the exchange joint components are bonded. For the same reason, a crescent-shaped mandrel 98 is inserted into the inflation lumen 84. With the guidewire lumen 54 and the mandrels 96 and 98 in place, the distal shaft 92 is slid around the exchange joint distal end 86 and heat shrink is wrapped around the joined components. The assembly is then heated, and the heat coupled with compression force from the heat shrink bonds the exchange joint distal end 86 to the distal shaft 92 and to the guidewire lumen 54. The mandrels are removed, and the exchange joint distal end 88 is then inserted into the proximal shaft 90. The exchange joint 80 and the proximal shaft 90 are bonded using heat and compression force from heat shrink wrapped around the assembly.

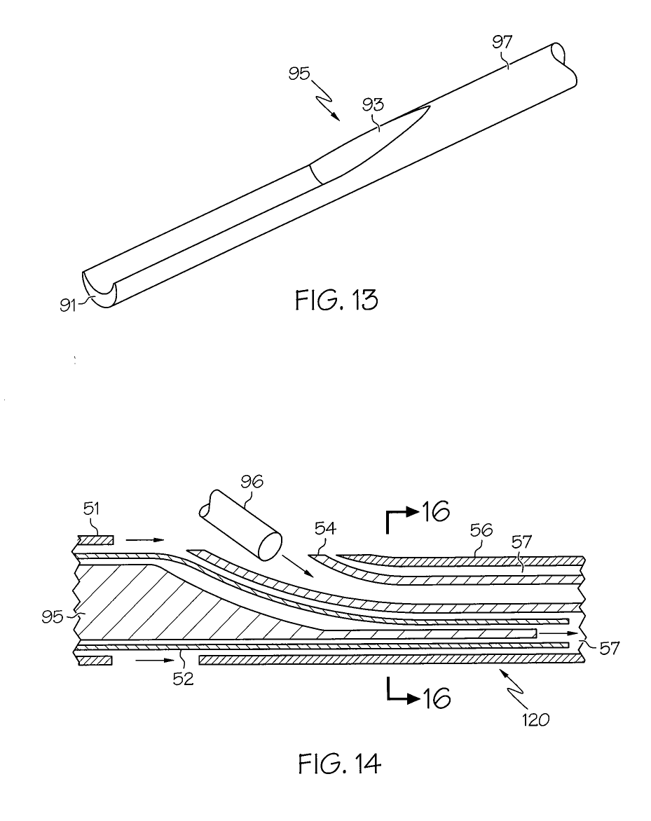

An exemplary crescent-shaped mandrel 98 such as that depicted in FIG. 15 has a crescent-shaped cross-section for a sufficient length of the mandrel 98 to maintain the inflation lumen's shape, particularly approaching the exchange joint's distal end 86 where the guidewire port 82 is larger and takes more space in the exchange joint 80. As best seen in FIG. 6, the inflation lumen 84 substantially consists of continuously formed first and second arced walls 85 and 87. The first wall 85 has a smaller radius of curvature than the second wall 87, and is arced to partially encircle the guidewire port 82. The outer surface 87 is also arced, and the two surfaces 85 and 87 create a crescent shape that gives the inflation lumen a large flow area while minimizing the exchange joint's longitudinal profile at the exchange joint distal end 86. Further, with the inflation lumen 84 gradually forming a crescent shape from the exchange joint proximal end 88 to the distal end 86 as the guidewire port 82 becomes increasingly entrenched in the exchange joint 80, the overall exchange joint is able to be formed with a small and uniform outer diameter. The mandrel 98 may have a crescent-shaped cross-section for all or most of the mandrel length. FIG. 13 is a perspective view of another exemplary mandrel 95 that has a crescent-shaped first end 91 and a stepped portion 93 that transitions the crescent shaped portion into a round portion 97. The stepped portion 93 and the round portion 97 support a substantial amount of the inflation lumen 84 when the exchange joint 80 is bonded to the guidewire lumen 54 and the distal shaft 92.

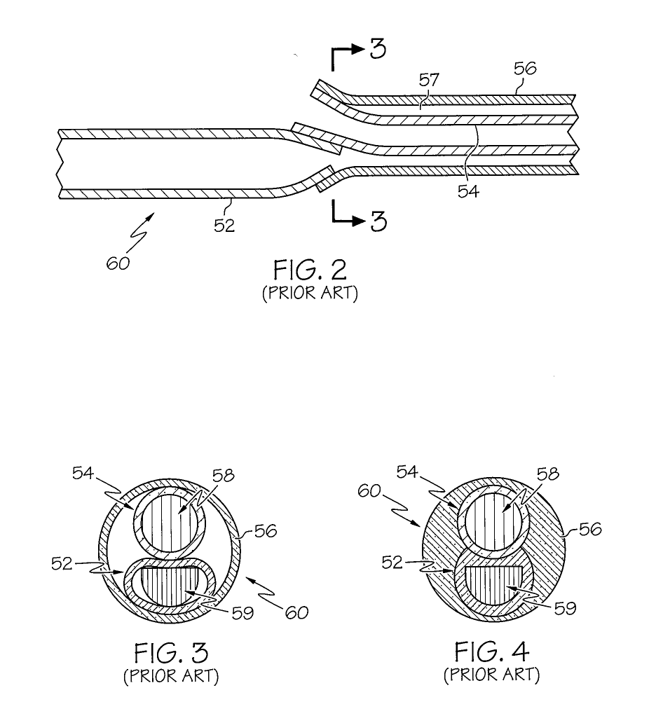

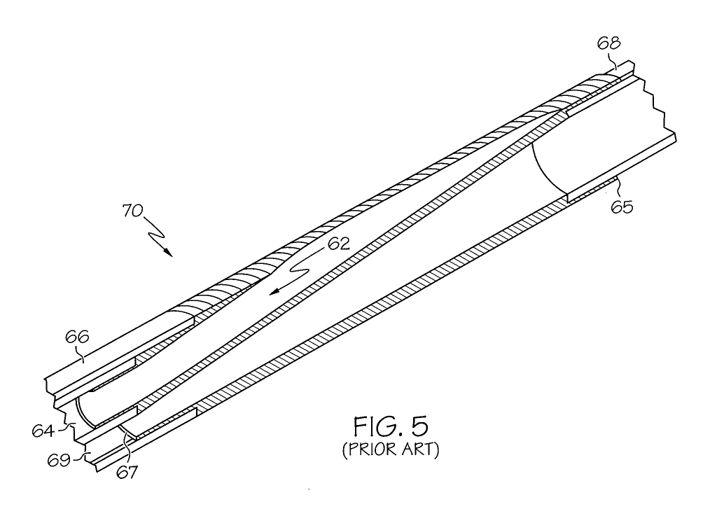

A crescent-shaped mandrel such as the mandrel 95 depicted in FIG. 13 is also useful when performing a multi-component exchange joint using a process similar to that previously discussed in connection with FIGS. 2 to 4. According to an exemplary method, an RX catheter exchange joint 120 depicted in FIG. 14 is assembled to include an elongate distal shaft 56 joined to transition tubing 52. The distal shaft 56 includes a coaxial inner guidewire lumen 54 extending to the shaft distal end. The transition tubing 52 joins the distal shaft 56 to a proximal shaft 51, which may optionally include or function as an inflation lumen through which a fluid is transported. FIG. 14 is a cross-sectional longitudinal view illustrating how the distal shaft 56 and the transition tubing 52 are joined. As depicted, the transition tubing 52 is inserted into the distal shaft 56. The guidewire lumen 54 is situated alongside the transition tubing at the position where the transition tubing 52 is inserted. During use, the transition tubing 52 transports fluid from the proximal shaft 51 to a distal shaft inflation lumen 57 that is coaxial with the guidewire lumen 54. Thus, the exchange joint 120 effectively transitions the inflation and guidewire lumens into the distal shaft 56 from a proximal side-by-side arrangement to a distal coaxial arrangement.

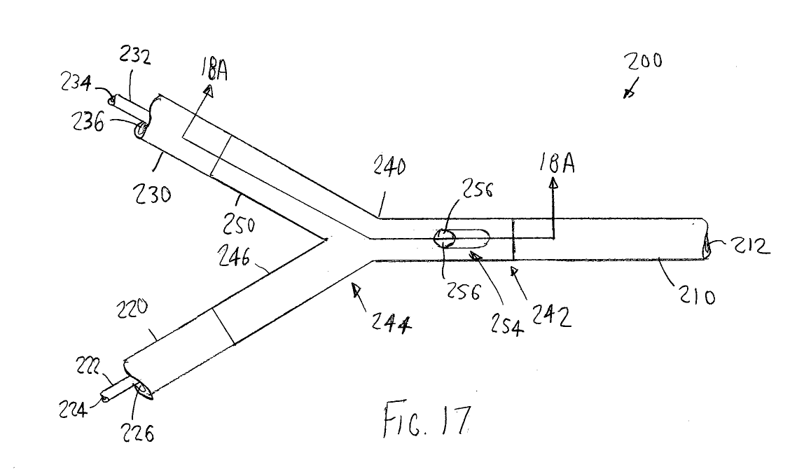

Assembly of the exchange joint 120 includes inserting the transition tubing 52 into the distal shaft 56. The inner diameter of the distal shaft 56 may need to be flared to allow room for the transition tubing 52, which also may require skiving. A round mandrel 96 is inserted into the lumen 54. Likewise, the crescent-shaped mandrel 95 is inserted into the transition tubing 52. FIG. 16 is a cross-sectional view of the exchange joint 120 taken along line 18-18 in FIG. 14 after performing a bonding procedure, with the crescent-shaped mandrel 95 loaded into the transition tubing 52 and the round mandrel 96 loaded into the guidewire lumen 54. The bonding process includes wrapping heat shrink material around the exchange joint 120. Heat is then applied to the exchange joint 120 as the heat shrink material compresses the joint components and brings the joint 120 to the bonded form depicted in FIG. 16. After the bonding process is completed, the mandrels 95 and 96 are removed and the lumen 54 is cut to form the guidewire entrance port.

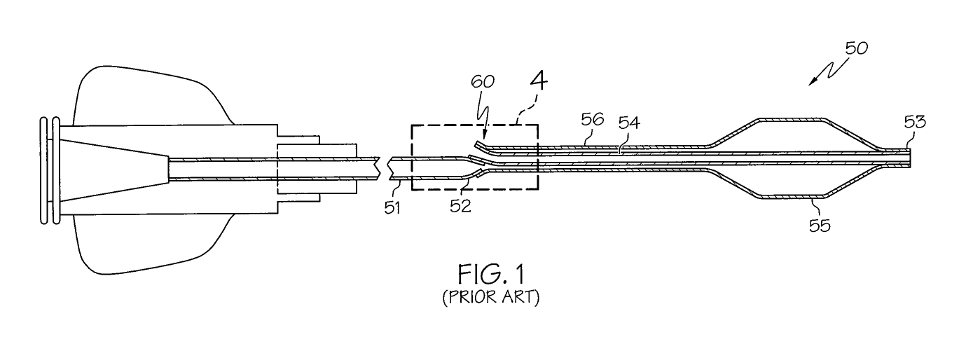

As previously discussed, the prior art RX catheter has an overall distinctively stepped shape at the exchange joint, as seen when viewing the joint 60 in FIG. 1. The step in the exchange joint 60 adds bulk to the shaft diameter, which may detrimentally affect catheter performance. More particularly, if the RX catheter 50 exits the distal end of a guide catheter, the step in the exchange joint 60 may become caught on the guide catheter edge while withdrawing the RX catheter from the vessel. Unlike the prior art assembly, the present exchange joint assembled using the crescent-shaped mandrel 95 has a substantially uniform outer diameter, as seen when viewing FIG. 14.

Using either of the above processes, an RX catheter having a unitary or a multi-component exchange joint may be manufactured. Each of the exchange joints includes an inflation lumen that transitions between a substantially round cross section to a crescent-shaped cross section in order to maintain a substantially uniform cross section from one end of the joint to the other. Although each joint provides different advantages, the unitary exchange joint 80 depicted in FIG. 6 provides the particular advantage of a ready-made joint that does not require flaring or skiving to combine the various lumens. Further, the unitary exchange joint 80 provides a convenient guidewire port 82 that gradually steers a guidewire toward and into the RX catheter distal end. The unitary exchange 80 joint may be a flexible component, and preferably has elasticity similar to that of both the proximal and distal shafts to which it is attached. Various moldable biocompatible polymers may be used to mold the unitary exchange joint 80 including polyamides, blends of polyamides and polyolefins, liquid crystal polymers, polyesters, polyketones, polyimides, polysulphones, polyoxymethylenes, polycarbonate, polymethyl methacrylate, polyolefins, cross-linked polyolefins, grafted polyolefins and other compatibilizers based on polyolefins. Lubrication additives may be included such as polyethylene micro-powders, fluoropolymers, silicone-based oils, fluoro-ether oils, molybdenum disulphide, graphite, and polyethylene oxide. Reinforcing additives may also be included, such as nano-clays, carbon fibers, and glass fibers or spheres. In addition, the unitary exchange joint 80 may be manufactured from harder and/or stiffer materials including biocompatible ceramics and biocompatible metals such as stainless steel.

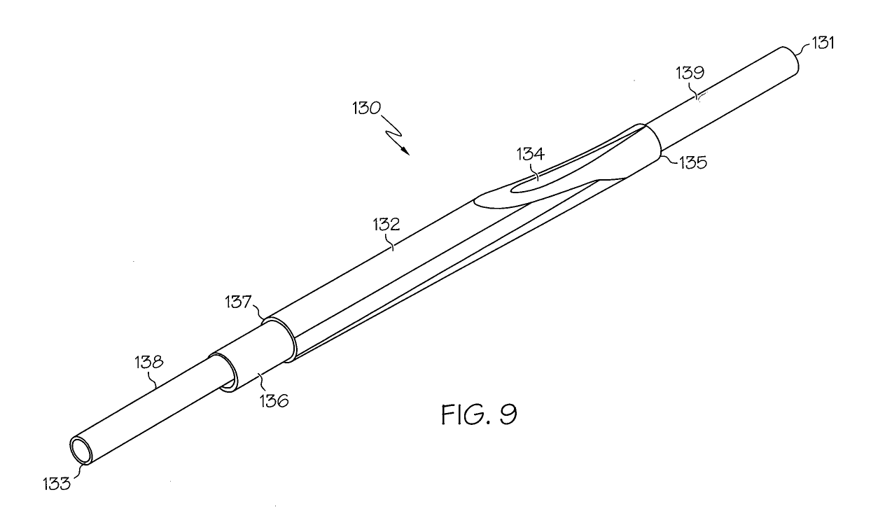

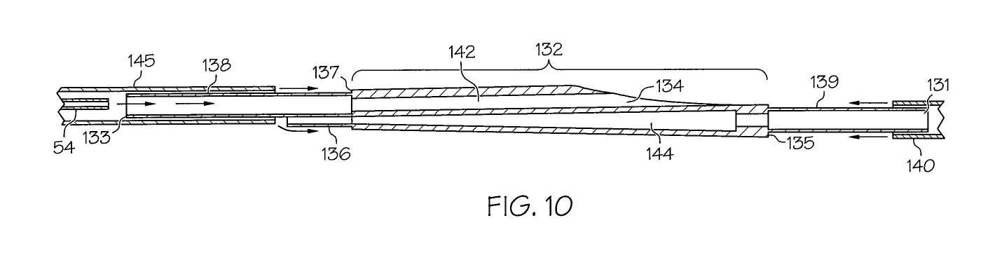

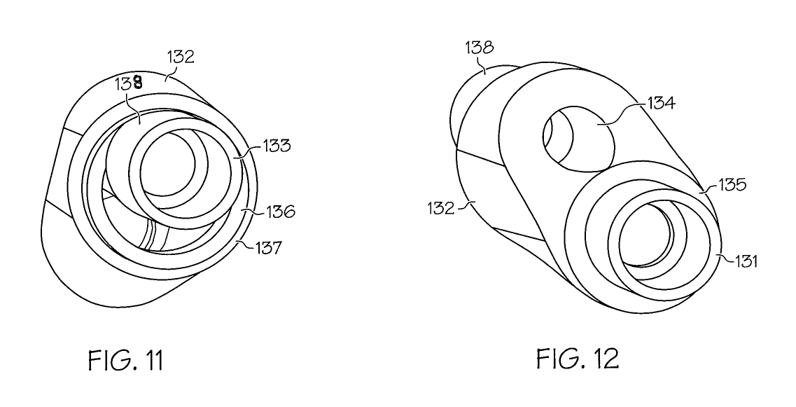

Turning now to FIGS. 9 to 12, another exemplary exchange joint 130 is depicted. FIGS. 9, 11, and 12 are perspective views of the exchange joint 130 at different angles. FIG. 11 is a perspective view of the exchange joint 130 taken from the joint distal end 133, and FIG. 12 is a perspective view taken from the joint proximal end 131. The exchange joint 130 comprises a main body portion 132 that includes a guidewire port 134, a guidewire lumen 142, and an inflation lumen 144, which transitions from having a substantially round cross-section to a crescent-shaped cross-section. At the exchange joint proximal end, an elongate tube 139 having a circular cross-section extends from the main body portion 132. As seen from viewing FIG. 10, the elongate tube 139 is in communication with the inflation lumen 144 and aids in attaching the RX catheter proximal shaft 140 to the exchange joint 130. To attach the two, the RX catheter proximal shaft 140 slides over the elongate tube proximal end 131 until the proximal shaft 140 abuts the main body portion proximal end 135.

At the exchange joint distal end, an inner lumen 138 and an outer lumen 136 extend from the main body portion 132. As seen from viewing FIG. 10, the inner lumen 138 slidingly receives a guidewire lumen 54. When properly inserted, the hypotube abuts the main body distal end 137, exits the inner lumen distal end 133, and extends to the RX catheter distal tip. During use, a guidewire is inserted into the guidewire port 134, through the main body guidewire lumen 142 and then into the guidewire lumen 54. The outer lumen 136 may be defined in part by tubular extension from the exchange joint 130 or, as depicted in FIG. 11, by an arced wall that is an extension of the crescent-shaped inflation lumen 144. The outer lumen 136 further aids in attaching the RX catheter distal shaft 145 to the exchange joint 130. To join the two, the RX catheter distal shaft 145 slides over the outer lumen 136 until the distal shaft 145 abuts the main body portion distal end 137.

In FIG. 10, the exchange joint 130 is depicted as a unitary assembly including the main body portion 132, the elongate tube 139, the inner lumen 138 and the outer lumen 136 molded as an integral exchange joint 130. However, the exchange joint 130 may also be assembled by manufacturing the elongate tube 139, the inner lumen 138 and the outer lumen 136 separately and then joining them together using a conventional process such as thermal bonding or UV cure bonding with adhesive. The exchange joint 130 may be a flexible component, and preferably has elasticity similar to that of both the proximal and distal shafts to which it is attached. Each component in the exchange joint 130 may be made using any of the materials previously listed with respect to the exchange joint 80 depicted in FIG. 6.

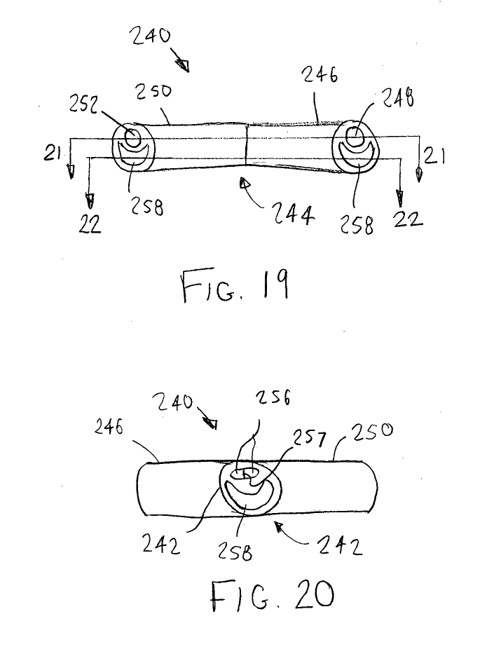

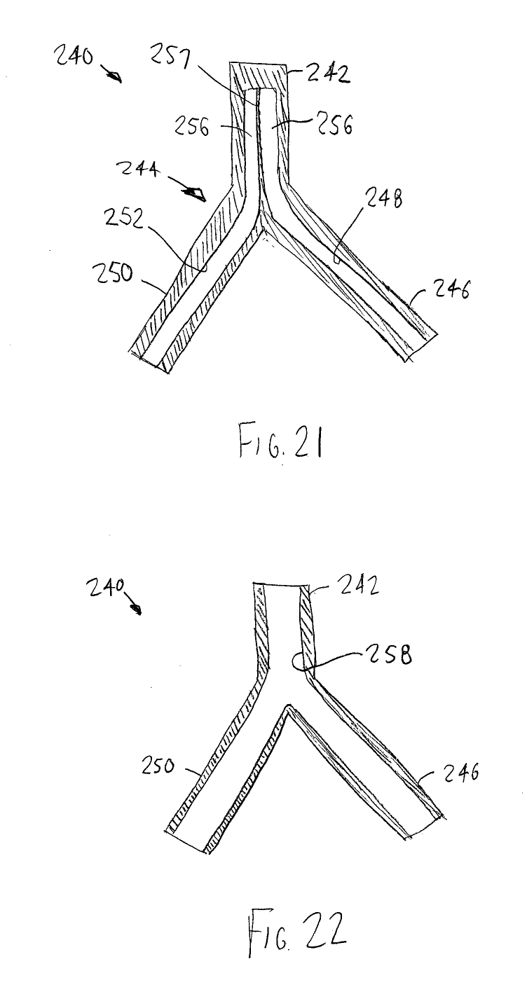

FIGS. 17-22 illustrate another embodiment of an RX catheter 200 according to the invention. As illustrated in FIG. 17, the RX catheter is a bifurcated catheter for bifurcated stent delivery, including but not limited to drug eluding bifurcated stent delivery.

In the illustrated embodiment, the catheter 200 includes a proximal shaft 210, a first distal outer shaft 220, a fist distal inner shaft 222, a second distal outer shaft 230, a second distal inner shaft 232, and an exchange joint 240 that is coupled between the proximal shaft 210 and the first and second distal outer shafts 220, 230 and inner shafts 222, 232. The proximal shaft 210 includes a proximal inflation lumen 212 that is in fluid communication with a fluid source that is configured to provide a fluid to the catheter 200. The fluid source may be of a conventional type and will not be discussed in further detail herein.

The first distal inner shaft 222 defines an inner lumen 224. The inner lumen 224 is configured to receive a guidewire (not shown). The first distal outer shaft 220 defines an outer inflation lumen 226 is configured to receive the fluid supplied by the fluid source and communicate the fluid to a balloon (not shown) that is located at or near a distal end of the first distal outer shaft 220. Similarly, the second distal inner shaft 232 defines an inner lumen 232. The inner lumen 234 is configured to receive a guidewire (not shown) at the same time a separate guidewire is received by the inner lumen 224 of the first distal inner shaft 222. This allows the catheter 200 to be inserted into different vessels at the same time. The second distal outer shaft 230 defines an outer inflation lumen 236 that is configured to receive the fluid supplied by the fluid source at the same time the outer inflation lumen 226 receives the fluid, and communicate the fluid to another balloon (not shown) that is located at or near a distal end of the second distal outer shaft 230. This allows the fluid to inflate both balloons at the same time for simultaneous stent delivery.

The exchange joint 240 includes a proximal end 242 that is configured to be coupled to the proximal shaft 210, and a distal end 244 that is configured to be coupled to the first distal shaft 220 and the second distal shaft 230. As shown in greater detail in FIGS. 19-21, the distal end 244 of the exchange joint 240 includes a first portion 246 that includes a first guidewire lumen 248, a second portion 250 that includes a second guidewire lumen 252. The exchange joint 240 also includes a guidewire port 254, shown in FIG. 17, which is configured to provide external access for a first guidewire into the first distal inner lumen 224 via the first guidewire lumen 248 and a second guidewire into the second distal inner lumen 234 via the second guidewire lumen 252. The guidewire port 254 begins just distal to the exchange joint proximal end 242 and gradually forms a deepening trench 256 in the exchange joint outer surface. A wall 257 may divide the deepening trench so that guidewires being inserted in the guidewire port 254 may be directed to the appropriate guidewire lumen 248, 252. At the exchange joint distal end 244, the guidewire port 254 almost entirely encloses two guidewires, and the wall defining the guidewire port 254 consequently has a C-shaped, nearly circular cross-section.

As shown in greater detail in FIGS. 19, 20, and 22, the exchange joint 240 also includes a transition lumen 258 that may be an inflation lumen that is in communication with the inflation lumen 212 of the proximal shaft 210, the outer inflation lumen 226 of the first distal outer shaft 220, and the outer inflation lumen 236 of the second distal outer shaft 230. The transition lumen 258 has a crescent-shaped cross-section at least at the first portion 246 and the second portion 250 of the distal end 244 of the exchange joint 240. The transition lumen 258 may also have a substantially round cross-section at the proximal end 242 of the exchange joint 240. The cross-sectional shape of the transition lumen 258 may wrap partially around the guidewire port 254 at least at the first portion 246 and the second portion 250 of the distal end 244 of the exchange joint 240.

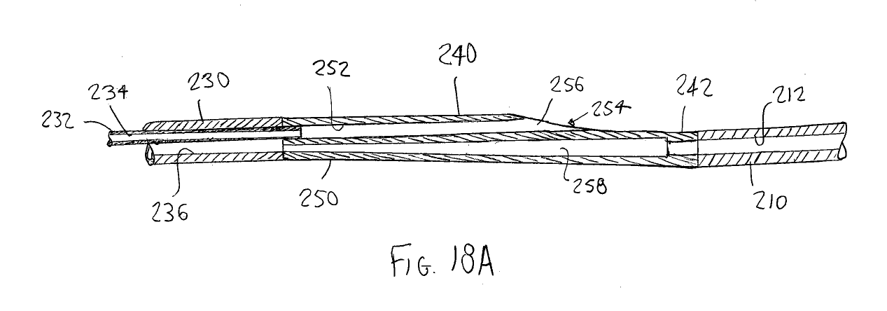

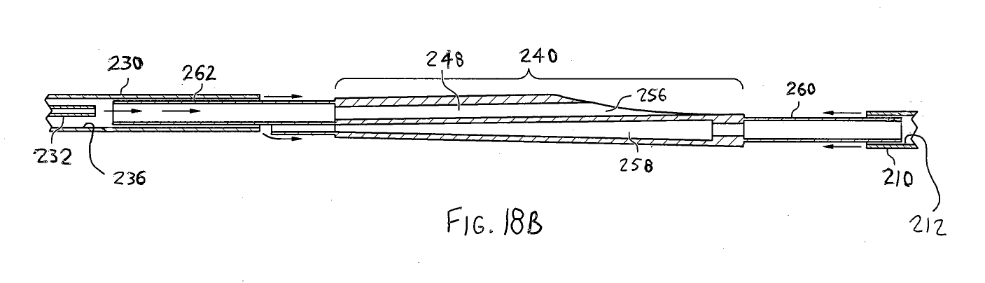

In the embodiment illustrated in FIG. 18A, the proximal shaft 210 and the first and second distal outer shafts 220, 230 and inner shafts 222, 232 may be connected to the exchange joint 240 by conventional methods, such as adhesive bonding or heat sealing, or may be insert molded with the exchange joint 240, as discussed in greater detail below.

In the embodiment illustrated in FIG. 18B, the rapid exchange catheter 200 may further include a proximal elongate tube 260, similar to the embodiment described above and illustrated in FIG. 10, that extends from the exchange joint 240 and is constructed and arranged to be in communication with the transition lumen 258 such that the elongate tube 260 joins the proximal shaft 210 to the exchange joint 240. The rapid exchange catheter 200 may also include a distal elongate tube 262 that extends from the distal end 244 of the exchange joint 240 and is in communication with the guidewire port 240. As illustrated, the distal elongate tube 262 is constructed and arranged to join the second distal inner shaft 232 and the second distal outer shaft 230 to the exchange joint 240. Similarly, the rapid exchange catheter 200 may further include a second distal elongate tube (not shown) that extends from the exchange joint distal end 244 and is in communication with the guidewire port 254 such that the second elongate tube 264 joins the first distal inner shaft 222 and the first distal outer shaft 220 to the exchange joint 240. As will be appreciated by one of ordinary skill in the art, the mandrels 96, 98 discussed above may be used in substantially the same manner described above to assist in assembling the rapid exchange catheter 200 and will not be described in further detail herein.

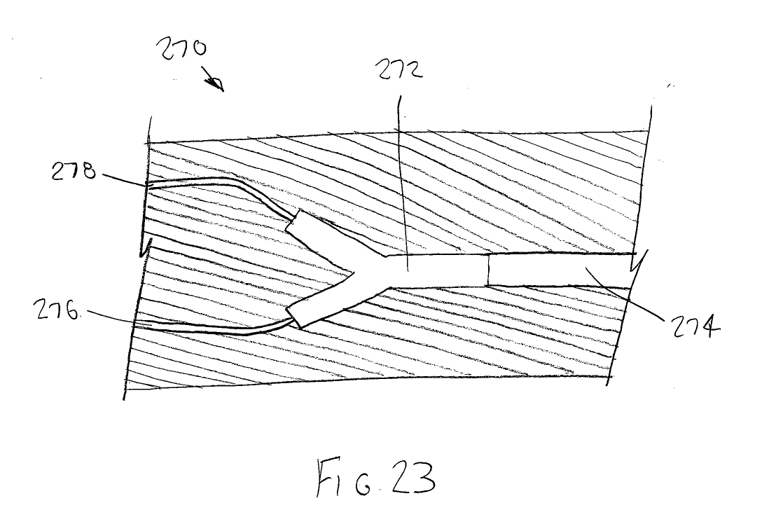

In an embodiment, the bifurcated stent delivery system 200 may be manufactured using the following process. First, as shown in FIG. 23, a mold 270 for insert molding the exchange joint 240 to the proximal shaft 220 and the first and second distal inner shafts 222, 232 is provided. The mold 270 includes an exchange joint cavity 272 that is configured to form the exchange joint 240 described above. The mold 270 also includes a proximal shaft receiving cavity 274 that is configured to receive the proximal shaft 210, a first distal inner shaft receiving cavity 276 that is configured to receive the first distal inner shaft 222, and a second distal inner shaft receiving cavity 278 that is configured to receive the second distal inner shaft 232. In some embodiments, portions of the exchange joint may already be formed and inserted into the exchange joint cavity 272, or the exchange joint 240 may be molded into a single piece. Once the proximal shaft 210 and the first and second distal inner shafts 222, 232 are placed in the mold 270 in their respective cavities 274, 276, 278, the exchange joint 240 may be molded.

Various moldable biocompatible polymers may be used to mold the exchange joint 240, including but not limited to polyamides, blends of polyamides and polyolefins, liquid crystal polymers, polyesters, polyketones, polyimides, polysulphones, polyoxymethylenes, polycarbonate, polymethyl methacrylate, polyolefins, cross-linked polyolefins, grafted polyolefins and other compatibilizers based on polyolefins. Lubrication additives may be included such as polyethylene micro-powders, fluoropolymers, silicone-based oils, fluoro-ether oils, molybdenum disulphide, graphite, and polyethylene oxide. Reinforcing additives may also be included, such as nano-clays, carbon fibers, and glass fibers or spheres.

After the proximal shaft 210, first distal inner shaft 222, and second distal inner shaft 232 have been insert molded to the exchange joint 240, the first distal outer shaft 220 may be attached to the first portion 246 of the distal end 244 of the exchange joint 240 so that the first distal outer shaft 220 surrounds the first distal inner shaft 222 and the first distal inflation lumen 226 communicates with the lumen 212 of the proximal shaft 210. The first distal outer shaft 220 may be attached by any suitable method, including but not limited to bonding. Similarly, the second distal outer shaft 230 may then be attached to the second portion 250 of the distal end 244 of the exchange joint 240 so that the second distal outer shaft 230 surrounds the second distal inner shaft 232 and the second distal inflation lumen 236 communicates with the lumen 212 of the proximal shaft 210. The above-described and illustrated embodiments are not intended to be limiting in any way. For example, features of the non-bifurcated RX catheters described above may also be incorporated in the bifurcated RX catheter 200, as would be appreciated by one of ordinary skill in the art.

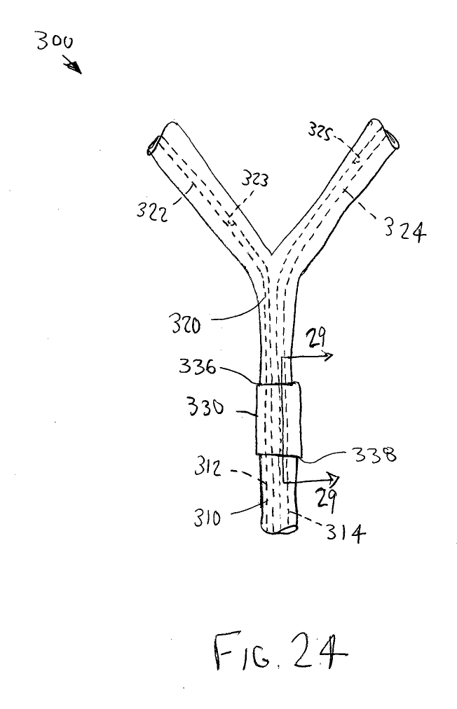

FIGS. 24-29 illustrate an over-the-wire bifurcated stent delivery system 300 according to embodiments of the invention. As shown in FIG. 24, the stent delivery system 300 includes a proximal shaft 310, a distal section 320, and a joint 330 that is coupled between the proximal shaft 310 and the distal section 320.

The proximal shaft 310 includes a first lumen 312 that is configured to receive a first guidewire, a second lumen 314 that is configured to receive a second guidewire, and a third lumen 316 that is configured to receive an inflation fluid. The third lumen 316 is configured to be in fluid communication with a fluid supply at a distal end of the proximal shaft 310.

The distal section 320 includes a first inner shaft 322, a second inner shaft 324, and an outer casing 326. The first inner shaft 322 defines a lumen 323 that is configured to receive the first guidewire, and the second inner shaft 324 defines a lumen 325 that is configured to receive the second guidewire. The outer casing 326 surrounds the first and second inner shafts 322, 324 and defines an inflation lumen 327 configured to receive the fluid from the fluid supply.

The joint 330 includes a proximal portion 332 and a distal portion 334. The distal portion 334 is configured to receive a proximal end 336 of the distal section 320, and the proximal portion 332 is configured to receive a distal end 338 of the proximal shaft 310 so that the first lumen 312 in the proximal shaft 310 is in communication with the lumen 323 of the first inner shaft 322, the second lumen 314 in the proximal shaft 310 is in communication with the lumen 325 of the second inner shaft 324, and the third lumen 316 of the proximal shaft 310 is connected to the outer inflation lumen 327 of the distal section 320.

As shown in FIG. 27, in an embodiment, the proximal portion 332 of the joint 330 includes a first insert 340 that is configured to be inserted into the first lumen 312 of the proximal shaft 310 and a second insert 342 that is configured to be inserted into the second lumen 314 of the proximal shaft 310. The proximal portion 332 is configured to sealingly engage the proximal shaft 310 so that fluid that is being supplied through the third lumen 316 does not leak at the joint 330.

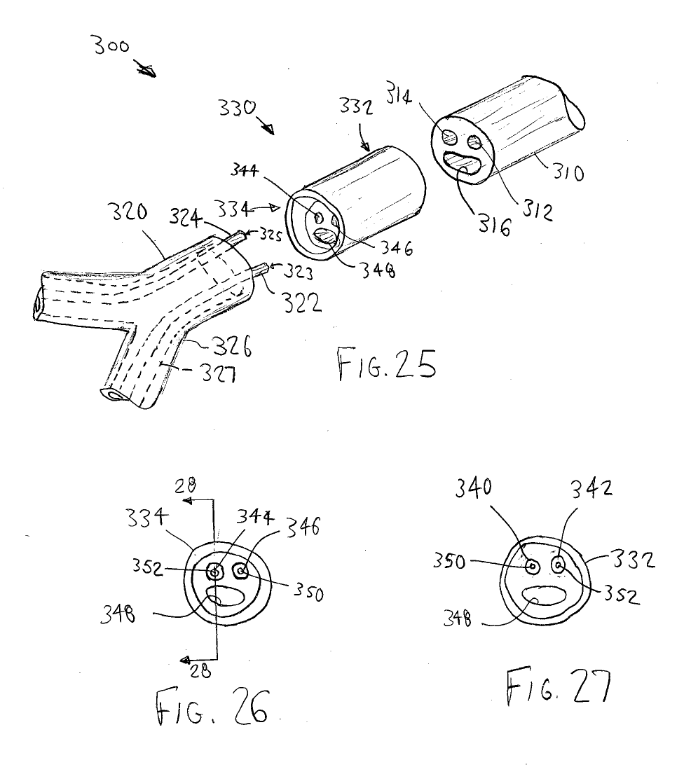

As shown in FIG. 26, in an embodiment, the distal portion 334 of the joint 330 includes a first port 344 that is configured to receive the first inner shaft 322 of the distal section 320, and a second port 346 that is configured to receive the second inner shaft 324 of the distal section 320. The distal portion 334 is also configured to sealingly engage the distal section 320 so that fluid that is being supplied to the outer inflation lumen 327 does not leak at the joint 330.

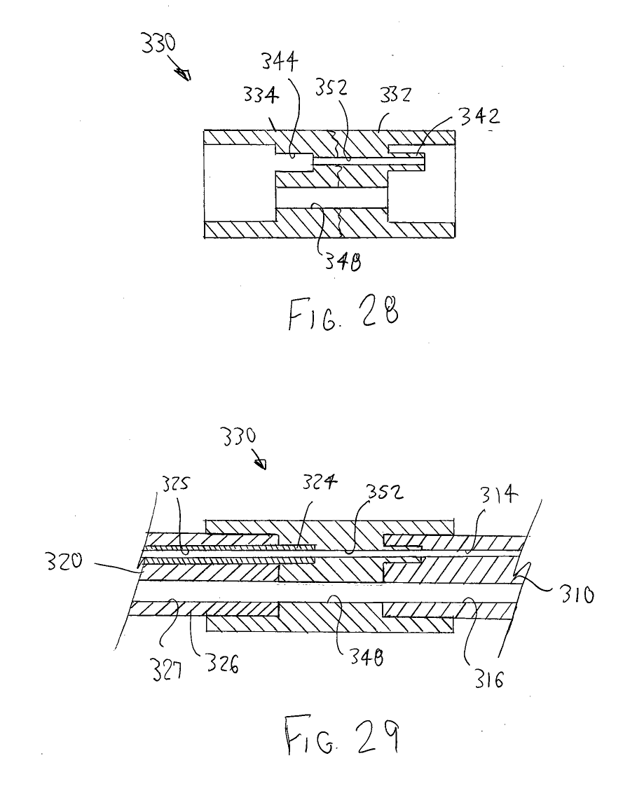

The joint 330 also includes an inflation lumen 348 that extends through both the proximal and distal portions 332, 334, as shown in FIGS. 28 and 29. The inflation lumen 348 is configured to allow the third lumen 316 of the proximal shaft 310 and the outer inflation lumen 327 of the distal section 320 to be in fluid communication with each other after the joint 330 is sealingly engaged with both the proximal shaft 310 and the distal section 320. The joint 330 further includes a first guidewire lumen 350 and a second guidewire lumen 352. The first guidewire lumen 350 is configured to allow the first guidewire to pass from the first lumen 312 of the proximal shaft 310 to the lumen 323 of the first inner shaft 322 of the distal section 320. Similarly, the second guidewire lumen 352 is configured to allow the second guidewire to pass from the second lumen 314 of the proximal shaft 310 to the lumen 325 of the second inner shaft 324 of the distal section 320, as will be appreciated from FIG. 29.

The proximal portion 332 of the joint 330 and the proximal shaft 310 may be formed from substantially the same material, including but not limited to polyethylene. Similarly, the distal portion 334 of the joint 330 and the distal section 320 may be formed from substantially the same material, including but not limited to polyamide, such as nylon, and polyether block amide, such as PEBAX®. Accordingly, the joint 330 may be molded such that the proximal portion 332 and the distal portion 334 are molded from two different materials, either simultaneously, or separately. In an embodiment, the proximal portion 332 and the distal portion 334 are molded separately, and then bonded together using a suitable method. In another embodiment, one of the portions may be molded first, and the other portion may be insert molded thereto. The illustrated embodiment is not intended to be limiting in any way. For example, in some embodiments, the entire joint 330 may be molded from a single material.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

What is claimed is:

1. A rapid exchange catheter, comprising:
a proximal shaft comprising a proximal inflation lumen;
a first distal shaft comprising a first distal inner lumen and a first distal outer inflation lumen;
a second distal shaft comprising a second distal inner lumen and a second distal outer inflation lumen; and
an exchange joint coupled between the proximal shaft and the first and second distal shafts, the exchange joint comprising
a proximal end configured to be coupled to the proximal shaft;
a distal end configured to be coupled to the first distal shaft and the second distal shaft, the distal end comprising
a first portion comprising a first guidewire lumen; and
a second portion comprising a second guidewire lumen; and
a guidewire port configured to provide access for a first guidewire into the first distal inner lumen via the first guidewire lumen and a second guidewire into the second distal inner lumen via the second guidewire lumen.

2. The rapid exchange catheter according to claim 1, wherein the exchange joint further comprises a transition lumen in communication with the proximal inflation lumen, the first distal outer lumen, and the second distal outer lumen, the transition lumen having a crescent-shaped cross-section at least at the first portion and the second portion of the distal end of the exchange joint.

3. The rapid exchange catheter according to claim 2, wherein the transition lumen has a substantially round cross-section at the proximal end of the exchange joint.

4. The rapid exchange catheter according to claim 2, wherein the transition lumen has a cross-sectional shape that wraps partially around the guidewire port at least at the first portion and the second portion of the distal end of the exchange joint.

5. The rapid exchange catheter according to claim 2, further comprising an elongate tube extending from the exchange joint and in communication with the transition lumen, the elongate tube joining the proximal shaft to the exchange joint.

6. The rapid exchange according to claim 2, wherein the transition lumen is an inflation lumen.

7. The rapid exchange catheter according to claim 1, further comprising a first elongate tube extending from the distal end of the exchange joint and in communication with the guidewire port, the first elongate tube joining the first distal inner lumen to the exchange joint.

8. The rapid exchange catheter according to claim 7, further comprising a second elongate tube extending from the exchange joint distal end and in communication with the guidewire port, the second elongate tube joining the second distal inner lumen to the exchange joint.

9. The rapid exchange catheter according to claim 1, wherein the exchange joint is an integrally molded structure.

10. The rapid exchange catheter according to claim 1, wherein the guidewire port forms a gradually deepening trench in the exchange joint.

11. A method for manufacturing a bifurcated stent delivery system, the method comprising:
inserting a proximal shaft into a first cavity of a mold, the proximal shaft comprising a lumen configured to communicate fluid;
inserting a first distal inner shaft into a second cavity of the mold, the first distal inner shaft defining a first guidewire lumen;
inserting a second distal inner shaft into a third cavity of the mold, the second distal inner shaft defining a second guidewire lumen;
insert molding an exchange joint to the proximal shaft, the first distal inner shaft, and the second distal inner shaft in a fourth cavity of the mold, the exchange joint comprising a proximal end, a distal end, and a guidewire port, the distal end comprising a first portion comprising a first lumen in communication with the first guidewire lumen, and a second portion comprising a second lumen in communication with the second guidewire lumen, the guidewire port being configured to provide access for a first guidewire into the first guidewire lumen and a second guidewire into the second guidewire lumen.

12. The method according to claim 11, further comprising
bonding a first outer distal shaft to the first portion of the distal end of the exchange joint so that the first outer distal shaft surrounds the first distal inner shaft and communicates with the lumen of the proximal shaft; and
bonding a second outer shaft to the second portion of the distal end of the exchange joint so that the second outer distal shaft surrounds the second distal inner shaft and communicates with the lumen of the proximal shaft.

13. An over-the-wire bifurcate stent delivery system, comprising:
a proximal shaft comprising a first lumen configured to receive a first guidewire, a second lumen configured to receive a second guidewire, and a third lumen configured to receive an inflation fluid;
a distal section comprising a first inner shaft defining a first guidewire lumen, a second inner shaft defining a second guidewire lumen, and an outer casing that surrounds the first and second inner shafts and defines an outer inflation lumen;
a joint coupled between the proximal shaft and the distal section, the joint comprising a proximal portion and a distal portion, the distal portion being configured to receive a proximal end of the distal section, and the proximal portion being configured to receive a distal end of the proximal shaft so that the first lumen in the proximal shaft is connected to the first guidewire lumen, the second lumen in the proximal shaft is connected to the second guidewire lumen, and the third lumen of the proximal shaft is connected to the outer inflation lumen.

14. The system according to claim 13, wherein the proximal portion comprises a first insert configured to be inserted into the first lumen of the proximal shaft and a second insert configured to be inserted into the second lumen of the proximal shaft.

15. The system according to claim 14, wherein the proximal portion is configured to sealingly engage the proximal shaft.

16. The system according to claim 13, wherein the distal portion comprises a first port configured to receive the first inner shaft of the distal section and a second port configured to receive the second inner shaft of the distal section.

17. The system according to claim 16, wherein the distal portion is configured to sealingly engage the distal section.

18. The system according to claim 13, wherein the proximal portion of the joint and the proximal shaft comprise substantially the same material.

19. The system according to claim 18, wherein the material comprises polyethylene.

20. The system according to claim 13, wherein the distal portion of the joint and the distal section comprise substantially the same material.

21. The system according to claim 20, wherein the material comprises nylon.

22. The system according to claim 20, wherein the material comprises polyether block amide.

23. A joint for coupling a proximal shaft to a distal section of an over the wire bifurcate stent delivery system, the proximal shaft comprising a plurality of lumens, the distal section comprising a plurality of lumens, the joint comprising:
a distal portion configured to receive a proximal end of the distal section; and
a proximal portion configured to receive a distal end of the proximal shaft so that a first lumen in the proximal shaft is connected to a first distal inner lumen in the distal section, a second lumen in the proximal shaft is connected to a second distal inner lumen in the distal section, and a third lumen of the proximal shaft is connected to an outer lumen in the distal section.

24. The joint according to claim 23, wherein the proximal portion comprises a first insert configured to be inserted into the first lumen of the proximal shaft and a second insert configured to be inserted into the second lumen of the proximal shaft.

25. The joint according to claim 24, wherein the proximal portion is configured to sealingly engage the proximal shaft.

26. The joint according to claim 24, wherein the distal portion comprises a first port configured to receive a first inner shaft of the distal section and a second port configured to receive a second inner shaft of the distal section.

27. The joint according to claim 26, wherein the distal portion is configured to sealingly engage the distal section.

28. The joint according to claim 23, wherein the proximal portion comprises substantially the same material as the proximal shaft.

29. The joint according to claim 28, wherein the material comprises polyethylene.

30. The joint according to claim 23, wherein the distal portion comprises substantially the same material as the distal section.

31. The joint according to claim 30, wherein the material comprises nylon.

32. The joint according to claim 30, wherein the material comprises polyether block amide.
